(19) United States
(12) Patent Application Publication
(10) Pub. No.: US 2025/0352053 A1
(43) Pub. Date: Nov. 20, 2025

(54) MONITORING METHOD AND SYSTEM FOR EYEBALL EMMETROPIZATION PROCESS

(71) Applicant: Shanghai Relative Space Time Life Technology Co., LTD, Shanghai (CN)

(72) Inventors: Jinling Yang, Shanghai (CN); Jianhua Xiao, Shanghai (CN); Xiaoyun Sun, Shanghai (CN); Xiaomei Qu, Shanghai (CN); Liangyong Xia, Shanghai (CN); Xingxue Zhu, Shanghai (CN)

(21) Appl. No.: 18/809,225

(22) Filed: Aug. 19, 2024

(30) Foreign Application Priority Data
May 14, 2024 (CN) 202410596505.2

(51) Int. Cl. A61B 3/00

(52) U.S. Cl. CPC A61B 3/0025

(57) ABSTRACT

The present application provides a monitoring method for an eyeball emmetropization process, including: acquiring eyeball data information about an eyeball to be monitored; outputting retinal optical characteristic data information; and analyzing and processing the retinal optical characteristic data information, and outputting an equation representing individual emmetropization characteristics as well as related parameters. Prompt is given in response to the abnormal trends detected in an eyeball refractive system that is in the developmental stage, controlling an eyeball emmetropization process within the normal range; and the degree and progress are monitored and analyzed for an eyeball with refractive errors, and the emmetropization trend and the variation in an axial length in the future are predicted.

### CROSS-REFERENCE TO RELATED APPLICATIONS

This application claims priority of Chinese Patent Application No. 202410596505.2, filed on May 14, 2024, the entire contents of which are incorporated herein by reference.

### TECHNICAL FIELD

The present application belongs to the technical field of medical data analysis, and specifically to a monitoring method and system for an eyeball emmetropization process.

### BACKGROUND

Eyeball emmetropization is a process of eyeball growth and refractive development, and the process is influenced by visual feedback related to the effective refractive status of the eyeball. In this process, the fine structure of the eyeball and internal and external environments form a dynamic system controlled by an active local signal feedback mechanism that regulates the eyeball emmetropization process in real time, so that the visual function of the eyeball meets the visual demand of an individual in the current internal and external environments.

Currently, the eyeball emmetropization process is mainly monitored by means of detecting individual's eye axial variation and refractive status, but neither of these two indicators can reflect the direction and degree of dynamic regulation in the eyeball emmetropization process in a timely manner. For example, if an adolescent's eye axis is found to be growing too fast, he is already deeply nearsighted; or if an adolescent is hard to see far clearly, he tends to be already nearsighted from the results of the optometry examination and the eye axis test. Patients with farsightedness have a slow growth rate of eye axis, resulting in hyperopic refractive error, which is also an abnormality in the eyeball emmetropization process. Regardless of nearsightedness or farsightedness, there is a need for a more sensitive and precise means to reflect the status and trend of the current eyeball emmetropization in a timely manner, so as to determine the progress and trend of nearsightedness or farsightedness, which facilitates the assessment of the efficacy of the current therapeutic means. This is important for the precise and individualized monitoring of eyeball emmetropization process as well as the development of individualized treatment plans for patients with refractive errors.

The above description is intended to provide general background information and does not necessarily constitute the prior art.

### SUMMARY

In response to the above drawbacks of the prior art, the present disclosure provides a monitoring method and system for an eyeball emmetropization process, for establishing a mathematical model on the basis of retinal optical characteristic data of an eyeball, and using an individualized assessment system to achieve accurate and individualized monitoring and prediction of the eyeball emmetropization process.

The present application provides a monitoring method for an eyeball emmetropization process, including: acquiring eyeball data information about an eyeball to be monitored; outputting retinal optical characteristic data information about the eyeball to be monitored on the basis of the eyeball data information about the eyeball to be monitored; and analyzing and processing the retinal optical characteristic data information and eye axial data information about the eyeball to be monitored, and outputting, for the eyeball to be monitored, an equation representing individual emmetropization characteristics as well as related parameters.

In an implementation of the present application, the acquiring eyeball data information about an eyeball to be monitored includes: measuring the eyeball to be monitored by means of a medical measuring instrument; storing the measured eyeball data information on the basis of user information about the eyeball to be monitored; and acquiring, from the stored eyeball data information, data information needs to be monitored currently about the eyeball to be monitored.

In an implementation of the present application, the outputting retinal optical characteristic data information about the eyeball to be monitored on the basis of the eyeball data information about the eyeball to be monitored includes the steps of: inputting the data information about the eyeball to be monitored into a preset mathematical model; and generating and outputting, after computational processing by the preset mathematical model, volumetric strain data and related values for a retinal photofocal plane or a retinal wavefront of the eyeball to be monitored.

In an implementation of the present application, the analyzing and processing the retinal optical characteristic data information about the eyeball to be monitored, and outputting, for the eyeball to be monitored, an equation representing individual emmetropization characteristics as well as related parameters includes the steps of: mathematically analyzing the volumetric strain data of the retinal photofocal plane or the retinal wavefront of the eyeball to be monitored and eye axial variation data, and obtaining the equation representing individual emmetropization characteristics as well as the related parameters; and monitoring and predicting the refractive development of the eyeball to be monitored on the basis of the equation representing individual emmetropization characteristics as well as the related parameters.

The present application further provides a monitoring method for an eyeball emmetropization process, including: acquiring measured data information about an eyeball to be monitored; processing the measured data information about the eyeball to be monitored, and outputting retinal optical characteristic data information about the eyeball to be monitored; and analyzing and processing the retinal optical characteristic data information and eye axial data information about the eyeball to be monitored, and outputting, for the eyeball to be monitored, an equation representing individual emmetropization characteristics as well as related parameters.

In an implementation of the present application, the acquiring measured data information about an eyeball to be monitored includes the steps of: measuring the eyeball to be monitored by means of a medical measuring instrument; storing the measured eyeball data information on the basis of user information about the eyeball to be monitored; and acquiring, from the stored eyeball data information, data information needs to be monitored currently about the eyeball to be monitored.

In an implementation of the present application, the outputting retinal optical characteristic data information about the eyeball to be monitored on the basis of the eyeball data information about the eyeball to be monitored includes the steps of: inputting the data information into a preset mathematical model; and generating and outputting, after computational processing by the preset mathematical model, volumetric strain and related values for a retinal photofocal plane or a retinal wavefront of the eyeball to be monitored.

The present application further provides a monitoring system for an eyeball emmetropization process, including: an acquisition module, configured to acquire eyeball data information about an eyeball to be monitored; a calculation module, configured to calculate retinal optical characteristic data information about the eyeball to be monitored on the basis of the eyeball data information; an analysis module, configured to analyze and process the retinal optical characteristic data information to obtain an equation representing individual emmetropization characteristics as well as related parameters, and monitor and predict the refractive development of an individual on the basis of the equation representing individual emmetropization as well as the related parameters; and an output module, configured to output the results of monitoring and predicting the refractive development of the eyeball to be monitored.

The present application further provides a computer-readable storage medium having at least one computer program stored thereon. The computer program is employed to implement the monitoring method for an eyeball emmetropization process as described above.

The present application further provides a computer program product, which, when runs on a computer, causes the computer to implement the monitoring method for an eyeball emmetropization process as described above.

As described above, the present application has the following beneficial effects by using the technical solutions disclosed therein.

1) Prompts are given timely in response to the abnormal trends detected in children or adolescents' eyeball refractive development that is in the developmental stage, so that timely interventions can be taken to control the eyeball emmetropization process within the normal range.

2) For children or adolescents with refractive errors (including nearsightedness and farsightedness), the system serves to monitor and analyze the degree and progress of refractive errors (including nearsightedness and farsightedness), to predict eyeball emmetropization trend and the axial length variation in the future, and to assess the interventions applied to the current refractive errors.

### BRIEF DESCRIPTION OF THE DRAWINGS

To state the technical solutions of the embodiments in the present application clearer, the attached drawings needed in the description of the embodiments are briefly introduced below. Obviously, the drawings described below are merely some embodiments in the present application, and for those ordinary skilled in the art, other drawings can be obtained according to these drawings without creative efforts.

FIG. 1 shows a flowchart of a monitoring method for an eyeball emmetropization process according to an embodiment of the present application.

FIG. 2 shows a flowchart of acquiring data information about an eyeball to be monitored according to an embodiment of the present application.

FIG. 3 shows a flowchart of analyzing volumetric strain according to an embodiment of the present application.

FIG. 4 shows a flowchart of a monitoring method for an eyeball emmetropization process according to an embodiment of the present application.

FIG. 5 shows a schematic structural diagram of a monitoring system for an eyeball emmetropization process according to an embodiment of the present application.

FIG. 6 shows a schematic diagram of a linear relation between volumetric strain of a retinal photofocal plane (reference control) and eye axial variation at the next time point of a nearsighted person according to an embodiment of the present application.

FIG. 7 shows a schematic diagram of a linear relation between volumetric strain of a retinal photofocal plane (reference control) and eye axial variation at the next time point of a farsighted person according to an embodiment of the present application.

FIG. 8 shows a schematic diagram of a linear relation between volumetric strain of a retinal photofocal plane (self control) and eye axial variation at the current time point of a nearsighted person according to an embodiment of the present application.

FIG. 9 shows a schematic diagram of a linear relation between volumetric strain of a retinal photofocal plane (self control) and eye axial variation at the current time point of a farsighted person according to an embodiment of the present application.

FIG. 10 shows a schematic diagram of a linear relation between volumetric strain of a retinal wavefront and eye axial variation at the next time point of a nearsighted person according to an embodiment of the present application.

FIG. 11 shows a schematic diagram of a linear relation between volumetric strain variation of a retinal wavefront and eye axial variation at the current time point of a nearsighted person according to an embodiment of the present application.

FIG. 12 shows a schematic diagram of a linear relation between volumetric strain of a retinal wavefront and eye axial variation at the next time point of a farsighted person according to an embodiment of the present application.

FIG. 13 shows a schematic diagram of a linear relation between volumetric strain variation of a retinal wavefront and eye axial variation at the current time point of a farsighted person according to an embodiment of the present application.

### DETAILED DESCRIPTION

Exemplary embodiments will be described herein in detail, examples of which are represented in the attached drawings. The same numerals in different attached drawings described below indicate the same or similar elements unless otherwise indicated. The implementations described in the following exemplary embodiments are merely examples of devices and methods consistent with some aspects of the present application as detailed in the appended claims, rather all implementations consistent with the present application.

It is to be noted that the terms “include”, “contain” or any other variations thereof are intended to cover non-exclusive inclusions, so that a process, method, object or device that includes a series of elements may include not only those elements, but also other elements not expressly listed, or also includes elements inherent to the process, method, object or device. In the absence of more limitations, the element defined by a sentence “including a . . . ” does not exclude that there are other identical elements in the process, method, object or device including the element. In addition, parts, features, and elements having the same name in different embodiments of the present application may have the same meaning or different meanings, and their specific meanings need to be determined as they are interpreted in the specific embodiment or further contextualized in the specific embodiment.

It is to be understood that although terms “first”, “second”, “third”, etc. are may employed herein to describe various information, the information is not limited to those terms, which are only used for distinguishing information with one type. For example, without departing from the scope of the present application, the first information also can be named as the second information, and vice versa. Depending on the context, the word “if” used herein can be interpreted as “in a case of”, “when” or “in response to the determination of”. Furthermore, as used herein, the singular forms “one”, “a” and “the” are intended to include the plural forms as well unless otherwise indicated. It is to be further understood that the terms “contain” and “include” indicate the presence of the described features, steps, operations, elements, components, items, species, and/or groups, but do not preclude the existence, presence, or addition of one or more other features, steps, operations, elements, components, items, species, and/or groups. The terms “or”, “and/or”, “including at least one of the following”, etc. used in the present application, may be construed to be inclusive or to mean any one or any combination thereof. For example, “including at least one of the following: A, B, C” means “any one of the following: A; B; C; A and B; A and C; B and C; A and B and C”; or “A, B or C” or A, B and/or C” means “any one of the following: A; B; C; A and B; A and C; B and C; and A and B and C”. Exceptions to this definition occur only when combinations of components, functions, steps, or operations are inherently mutually exclusive in certain ways.

It is to be understood that although various steps in the flowchart of the embodiment of the present application are shown sequentially as indicated by the arrows, they are not necessarily executed sequentially in the order indicated by the arrows. Unless expressly stated herein, there is no strict limitation on the order of the execution of these steps, which can be executed in other orders. Moreover, at least a portion of the steps in the drawings may include a plurality of sub-steps or a plurality of phases, which are not necessarily executed at the same moment, but may be at different moments, and the order in which these sub-steps or phases are executed is not necessarily sequential but may be rotated or alternately executed with at least a portion of the other steps or sub-steps or phases of the other steps.

Depending on the context, the word “if” used herein can be interpreted as “in a case of”, “when”, “in response to the determination of” or “in response to the detection of”. Similarly, depending on the context, the phrases “if determined” or “if detected (the stated condition or event)” can be interpreted as “when determined” or “in response to the determination of” or “when detected (the stated condition or event)” or “in response to the detection of (the stated condition or event)”.

It is to be noted that in this paper, step designations such as S1 and S2 are adopted for merely expressing the corresponding contents more clearly and briefly, rather than constituting a substantive limitation on the order. A person skilled in the art may execute S4 before S3, etc., in the specific implementation, which is within the scope of protection of the present application.

It is to be understood that the specific embodiments described herein are merely illustrative of the present application rather than limiting the scope of the present application.

In the ensuing description, the terms such as “module”, “part”, or “unit” used to denote components are merely for the ease in description of the present application and have no specific meanings. Thus, the “module”, “part” or “unit” can be used interchangeably.

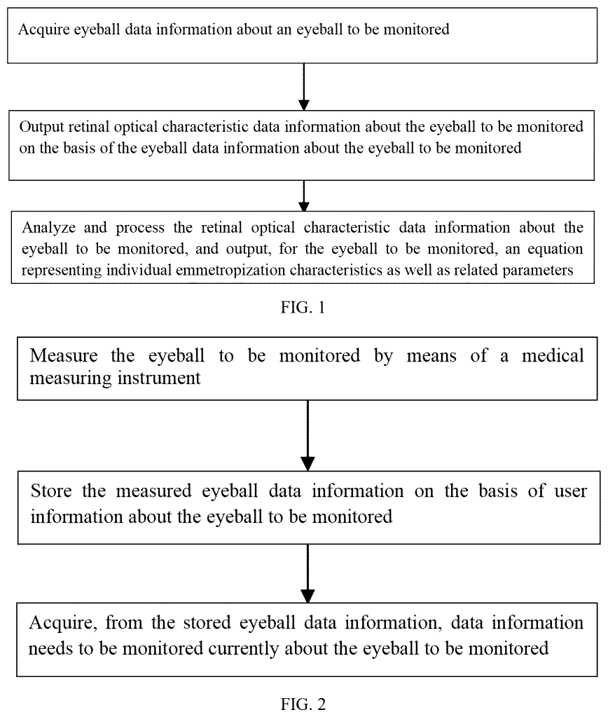

Referring to FIG. 1, the present application provides a monitoring method for an eyeball emmetropization process, including the following steps. Eyeball data information about an eyeball to be monitored is acquired; retinal optical characteristic data information about the eyeball to be monitored is outputted on the basis of the eyeball data information about the eyeball to be monitored; and the retinal optical characteristic data information about the eyeball to be monitored is analyzed and processed, and an equation representing individual emmetropization characteristics as well as related parameters are outputted for the eyeball to be monitored.

In an embodiment of the present application, it is first necessary to acquire data information about the user's eyeball to be monitored in monitoring the emmetropization process, such as biological indicators of the eyeball including axial length, corneal thickness, corneal curvature, anterior chamber depth, lens thickness, vitreous chamber length, and choroidal thickness in the central and peripheral regions, as well as pupil diameter, and Kappa angle; and such as optical indicators of the user's eyeball including wavefront aberration (including low-order and high-order aberration), focal power (refraction), relative refraction (subtracting central refraction from peripheral refraction), and volumetric strain of the eyeball's retinal imaging in the center and peripheral regions of the eyeball. On the basis of the obtained data of the eyeball to be monitored, retinal optical characteristic data information about the eyeball to be monitored is calculated, such as volumetric strain data and related values of a retinal photofocal plane or a retinal wavefront of the eyeball in the center and peripheral regions. Further, the retinal optical characteristic data information about the eyeball to be monitored is analyzed and processed, to ultimately obtain the emmetropization degree of the eyeball to be monitored, such as: an equation representing individual emmetropization characteristics as well as related parameter information.

Referring to FIG. 2, in a solution of the present application, acquiring eyeball data information about an eyeball to be monitored includes the following steps. The eyeball to be monitored is measured by means of a medical measuring instrument; the measured eyeball data information is stored on the basis of user information about the eyeball to be monitored; and data information needs to be monitored currently about the eyeball to be monitored is acquired from the stored eyeball data information.

Users, whose eyeball emmetropization needs to be monitored, need to have data information collected regularly on the aforementioned biological indicators and optical indicators of the eyeball. In an embodiment of the present application, the corresponding data of the eyeball to be monitored is measured by a medical measuring instrument, which, after the measurement, is stored according to user information, such as name, age and gender, so as to facilitate easy access to the eyeball data information of the user to be monitored at various time points when they are analyzed and processed subsequently. The user may measure the eyeball data using other measuring instruments, resulting in the measured eyeball data failing to be directly stored. Accordingly, in another embodiment of the present application, the measured printed data information is scanned, from which, relevant data information is extracted and stored according to the user.

In an implementation of the present application, the outputting retinal optical characteristic data information about the eyeball to be monitored on the basis of the eyeball data information about the eyeball to be monitored includes the following steps. The eyeball data information is inputted into a preset mathematical model; and after computational processing by the preset mathematical model, volumetric strain and related values for a retinal photofocal plane or a retinal wavefront of the eyeball to be monitored are generated and outputted.

In an embodiment of the present application, the obtained eyeball data information about the user to be monitored is subjected to computational processing by means of the preset mathematical model, which is as follows.

$${ε}_{w} ( φ , θ ; T ) = \frac{1}{3} \{ 3 {\overline{H}}_{1} ( φ ; 0 ) \cdot [ h ( φ , θ ; T ) ] + {\overline{H}}_{2} ( φ ; 0 ) \cdot {[ h ( φ , θ ; T ) ]}^{2} \}$$

where ϵ_{w} represents volumetric strain of a retinal photofocal plane or a retinal wavefront of an eyeball, H₁ represents an average curvature of optical imaging of an eyeball, H₂ represents a Gaussian curvature of optical imaging of an eyeball, h represents an elevation difference of optical imaging of an eyeball, φ and θ represent a latitude and a longitude in the retinal photofocal plane or the retinal wavefront, respectively, and T represents a time point.

After computational processing by the preset mathematical model, the volumetric strain for the retinal photofocal plane or the retinal wavefront of the user's eyeball to be monitored is obtained.

In an embodiment of the present application, it is preferentially necessary to optimize the acquired data of the eyeball to be monitored, before being subjected to computational processing by the mathematical model to obtain the volumetric strain of the retinal photofocal plane or the retinal wavefront. For example:
the curvature value calculated using the total spherical equivalent (diopter of spherical power+½ diopter of cylindrical power) of a person's eyeball to be monitored is taken as the average curvature (H₁);
the square of the curvature value calculated from the total diopter of spherical power of the person's eyeball to be monitored is taken as the Gaussian curvature (H₂);
the aberration value of the person's eyeball to be monitored is taken as the elevation difference (h); and
the curvature is calculated, by the formula for focal power (refraction), D=n/f, on the basis of the refraction value or relative refraction value from the retinal imaging of the person's eyeball to be monitored. D represents the focal power (refraction); n represents a refractive index; and f represents a focal length, which is a radius of curvature in this system.

After the obtained eyeball data is optimized by the way described above, the processed results are sent to the preset mathematical model for computational processing.

The equation for the emmetropization characteristics of the eyeball to be monitored and the related parameters are outputted. In a preferred implementation, the assessment result of the emmetropization degree is sent directly via a mobile network to a mobile terminal of a user whose eyeball needs to be monitored, so as to facilitate the user to obtain the monitoring result of the eyeball at the first time.

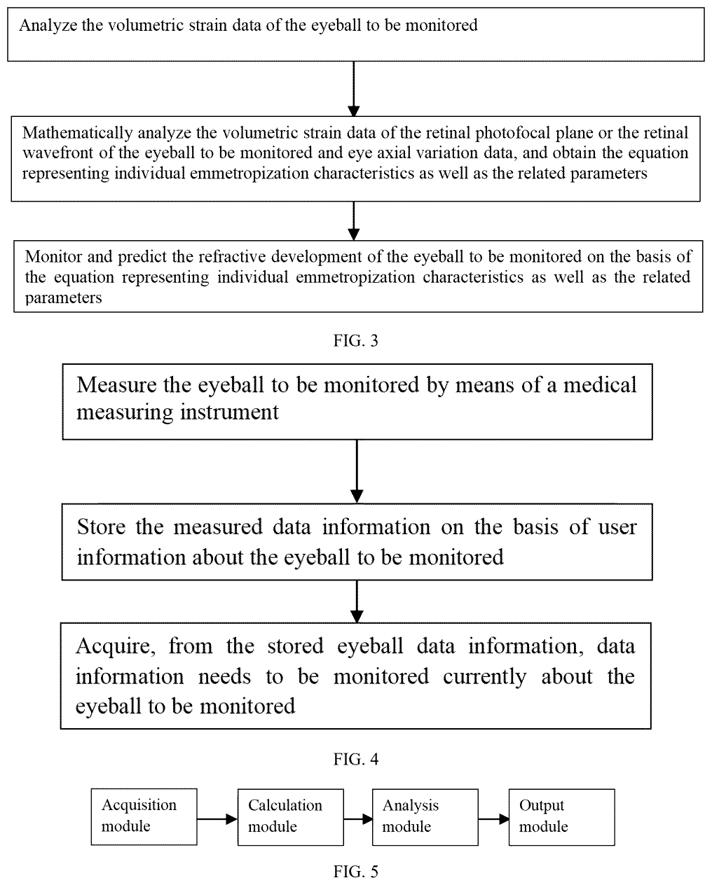

Referring to FIG. 3, in a solution of the present application, after the volumetric strain and related values of the retinal photofocal plane or the retinal wavefront of the eyeball to be monitored are obtained, the value of the volumetric strain is further analyzed. The volumetric strain data of the retinal photofocal plane or the retinal wavefront of the eyeball to be monitored and eye axial variation data are mathematically analyzed, and the equation representing individual emmetropization characteristics as well as the related parameters are obtained. The refractive development of an individual is monitored and predicted on the basis of the equation representing individual emmetropization as well as the related parameters.

Referring to FIG. 4, it shows a flowchart of a monitoring method for an eyeball emmetropization process provided by the present application. The monitoring method for an eyeball emmetropization process includes the following steps. Measured data information about an eyeball to be monitored is acquired; the measured data information about the eyeball to be monitored is processed, and retinal optical characteristic data information about the eyeball to be monitored is outputted; and the retinal optical characteristic data information about the eyeball to be monitored is analyzed and processed, and an equation representing individual emmetropization characteristics as well as related parameters are outputted for the eyeball to be monitored.

In an embodiment of the present application, in the case of monitoring emmetropization process of the user's eyeball, as described above, after the data about the user's eyeball to be monitored is obtained, such as the biological indicators of the eyeball including axial length, corneal thickness, corneal curvature, anterior chamber depth, lens thickness, vitreous chamber length, and choroidal thickness in the central and peripheral regions, as well as pupil diameter, and Kappa angle, and such as the optical indicators of the user's eyeball including wavefront aberration (including low-order and high-order aberration), focal power (refraction), and relative refraction (subtracting central refraction from peripheral refraction) of the eye's retinal imaging in the center and peripheral regions, the volumetric strain data for a retinal photofocal plane or a retinal wavefront of the eyeball to be monitored is calculated on the basis of the data of the eyeball to be monitored. Further, the volumetric strain data of the retinal photofocal plane or the retinal wavefront of the eyeball to be monitored and the eye axial variation data are mathematically analyzed, to obtain the equation representing individual emmetropization characteristics as well as the related parameters. In a solution of the present application, the refractive development of the eyeball to be monitored is monitored and predicted on the basis of the equation representing individual emmetropization characteristics as well as the related parameters.

With continued reference to FIG. 2, in a solution of the present application, acquiring data information about the eyeball to be monitored includes the following steps. The eyeball to be monitored is measured by means of a medical measuring instrument; the measured eyeball data information is stored on the basis of user information about the eyeball to be monitored; and from the stored eyeball data information, eyeball data information needs to be monitored currency about the eyeball to be monitored is acquired. In an embodiment of the present application, after the measured eyeball data is stored on the basis of the user information, such as name, age and gender, a reminder notification is provided for sending a measurement notification to the user to be monitored prior to the arrival of the date for measuring the eyeball data. In another embodiment of the present application, the measured eyeball data information after being obtained can be stored by means of manual input.

In an implementation of the present application, the outputting retinal optical characteristic data information about the eyeball to be monitored on the basis of the eyeball data information about the eyeball to be monitored includes the following steps. The eyeball data information is inputted into a preset mathematical model; and after computational processing by the preset mathematical model, volumetric strain and related values for a retinal photofocal plane or a retinal wavefront of the eyeball to be monitored are generated and outputted.

In an embodiment of the present application, the obtained data information about the user's eyeball to be monitored is subjected to computational processing by means of the preset mathematical model, which is as follows.

$${ε}_{w} ( φ , θ ; T ) = \frac{1}{3} \{ 3 {\overline{H}}_{1} ( φ ; 0 ) \cdot [ h ( φ , θ ; T ) ] + {\overline{H}}_{2} ( φ ; 0 ) \cdot {[ h ( φ , θ ; T ) ]}^{2} \}$$

where ϵ_{w} represents volumetric strain of optical imaging of an eyeball, H₁ represents an average curvature of optical imaging of an eyeball, H₂ represents a Gaussian curvature of optical imaging of an eyeball, h represents an elevation difference of optical imaging of an eyeball, φ and θ represent a latitude and a longitude in a retinal photofocal plane or a retinal wavefront, respectively, and T represents a time point.

After computational processing by the preset mathematical model, an analysis diagram of the volumetric strain for the retinal photofocal plane or the retinal wavefront of the user's eyeball to be monitored is obtained.

In an embodiment of the present application, it is preferentially necessary to optimize the acquired eyeball data, before being subjected to the computational processing by the mathematical model described above to obtain the volumetric strain of the retinal photofocal plane or the retinal wavefront. For example:
the curvature value calculated using the total spherical equivalent (diopter of spherical power+½ diopter of cylindrical power) of a person's eyeball to be monitored is taken as the average curvature (H₁);
the square of the curvature value calculated from the total diopter of spherical power of the person's eyeball to be monitored is taken as the Gaussian curvature (H₂);
the aberration value of the person's eyeball to be monitored is taken as the elevation difference (h); and
the curvature is calculated, by the formula for focal power (refraction), D=n/f, on the basis of the refraction value or relative refraction value from the retinal imaging of the person's eyeball to be monitored. D represents the focal power (refraction); n represents a refractive index; and f represents a focal length, which is a radius of curvature in this system.

In an implementation of the present application, a reference eye or a reference wavefront is taken as a control, and the linear equation formed by the volumetric strain of the peripheral or paracentral retinal photofocal plane or the retinal wavefront of an individual (reference control) obtained by means of the preset mathematical model and the eye axial variation at the next point time is defined as a driving force equation, which can serve to assess the magnitude of the driving force of the peripheral or paracentral retinal photofocal plane or retinal wavefront of the eyeball on the eye axial growth for the individual at the current stage, and to predict the eye axial variation for the individual at a future time point, as shown in Table 1.

TABLE 1
Volumetric strain and eye axial variation at the next time point

| | Eye axial | | | |
|---|---|---|---|---|
| | variation | Volumetric | Volumetric strain | Volumetric strain |
| | (next time | strain from | from aberrometer | from aberrometer |
| Data source | point) | MRT data (+) | data (+) | data (−) |
| 9-MZX-F-OD V0 | 0.51 | 1.3486 | | |
| 9-MZX-F-OD V1 | 0.13 | 0.4537 | 0.0422 | −0.0109 |
| 4-WZR-M-OS V0 | 0.27 | 0.6179 | | |
| 8-ZZM-M-OD V1 | −0.03 | 0.0624 | 2.0950 | −0.5074 |
| 8-ZZM-M-OS V1 | −0.12 | 0.4475 | 4.2673 | −0.9574 |
| 7-ZBN-M-OD V0 | 0.21 | 0.3008 | | |
| 7-ZBN-M-OS V0 | 0.12 | 0.2604 | | |
| 9-MZX-F-OS V1 | 0.03 | 0.0868 | 0.0391 | −0.0091 |
| 10-LWQ-F-OD V1 | −0.01 | 0.2423 | 0.0598 | −0.0151 |
| 10-LWQ-F-OS V1 | 0.08 | 0.1087 | 0.0101 | −0.0027 |

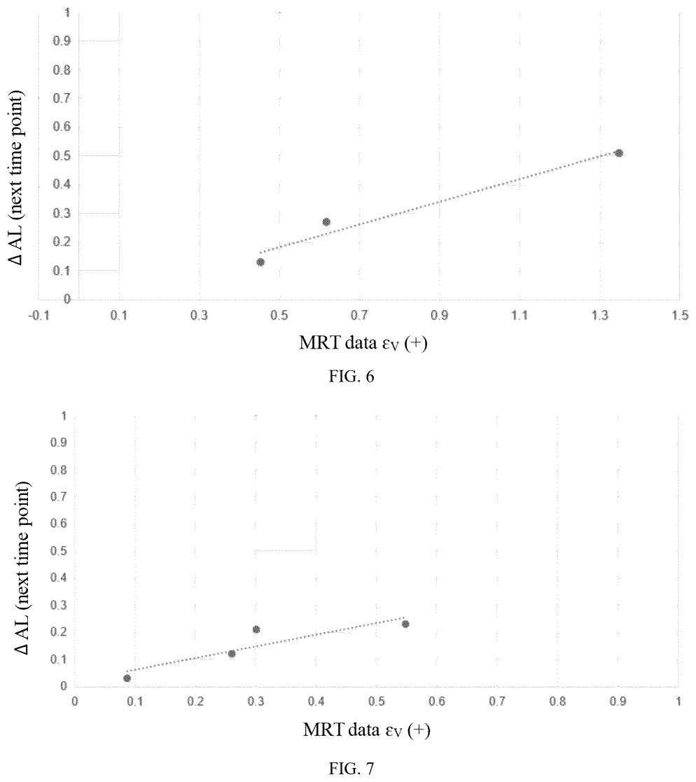

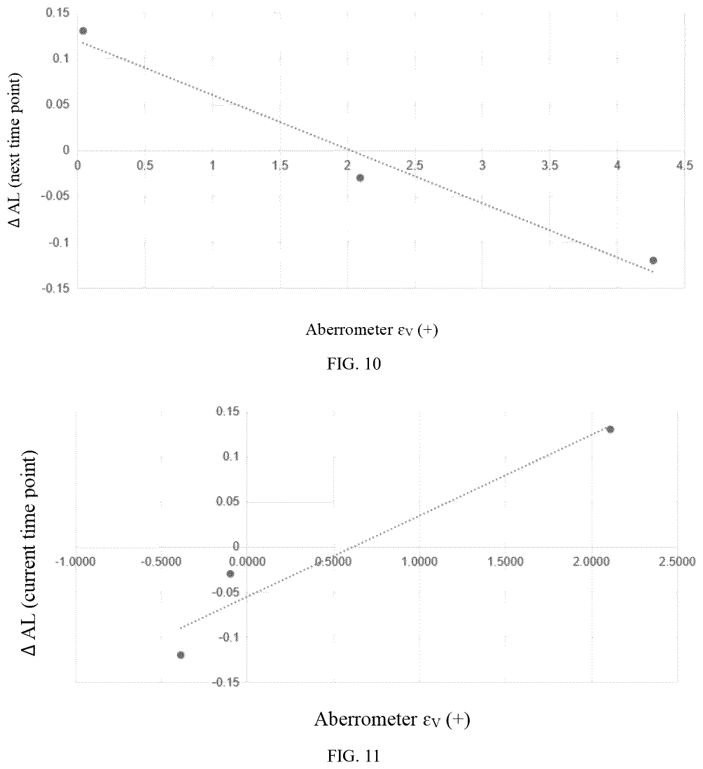

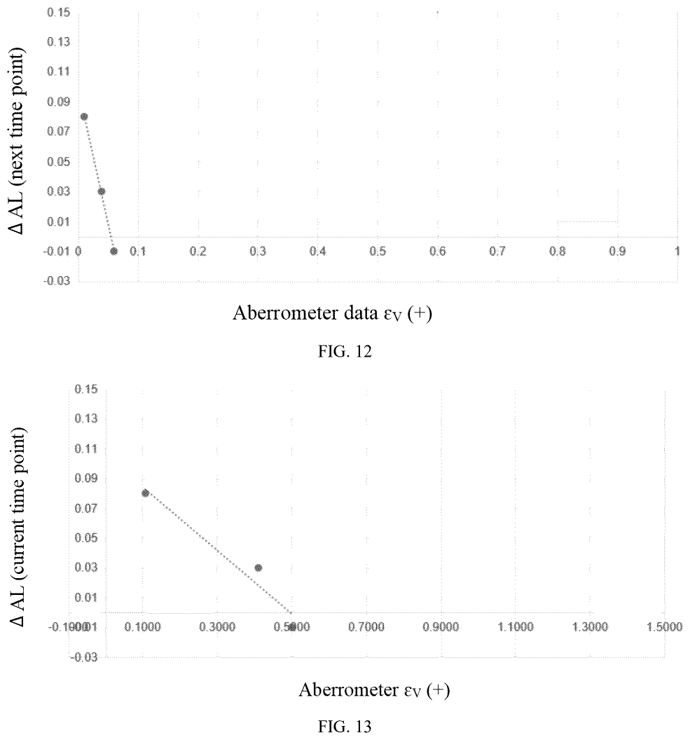

FIG. 6 shows a linear relation between volumetric strain of a retinal photofocal plane (reference control) and eye axial variation at the next time point of a nearsighted person. FIG. 7 shows a linear relation between volumetric strain of a retinal photofocal plane (reference control) and eye axial variation at the next time point of a farsighted person. FIG. 10 shows a linear relation between volumetric strain of a retinal wavefront and eye axial variation at the next time point of a nearsighted person. FIG. 12 shows a linear relation between volumetric strain of a retinal photofocal plane and eye axial variation at the next time point of a nearsighted person. For the driving force equation, the relatively smaller slope reflects the smaller driving force of the peripheral or paracentral retinal photofocal plane or retinal wavefront on the growth of the eye axis, and, conversely, the relatively larger slope reflects a larger driving force. As seen from the linear schematic diagrams of FIGS. 6, 7, 10, and 12, the driving force of a farsighted person is smaller than that of a nearsighted person.

In an embodiment of the present application, the data of the retinal photofocal plane or the retinal wavefront of an individual at a previous time point is taken as a control, and the linear equation formed by the volumetric strain of the peripheral or paracentral retinal photofocal plane (self control) or the variation in the volumetric strain of the retinal wavefront of the individual obtained by means of preset mathematical model and eye axial variation at the current time point is defined as a sensitivity equation, which can serve to assess the magnitude of the sensitivity of the peripheral or paracentral retinal photofocal plane or retinal wavefront on the eye axial growth at the current time point for an individual, as shown in Table 2.

TABLE 2
Volumetric strain (self control)/volumetric strain variation
and eye axial variation at current time point

| | | | Volumetric | |
|---|---|---|---|---|
| | Eye axial | Volumetric | strain variation | Volumetric strain |
| | variation | strain from | from | variation from |
| | (current time | MRT | aberrometer | aberrometer data |
| Data source | point) | data (self control) | data (+) | (−) |
| 9-MZX-F-OD V2 | 0.13 | 0.2994 | 2.1111 | −0.4715 |
| 6-LYC-F-OD V1 | 0.04 | 0.1046 | | |
| 6-LYC-F-OS V1 | 0.07 | 0.4184 | | |
| 2-ZSK-F-OD V1 | 0.24 | 0.2486 | | |
| 2-ZSK-F-OD V2 | 0.22 | 0.3380 | | |
| 2-ZSK-F-OD V3 | 0.14 | 0.5436 | | |
| 8-ZZM-M-OD V2 | −0.03 | 0.1134 | −0.0984 | 0.0155 |
| 8-ZZM-M-OS V2 | −0.12 | 0.0933 | −0.3863 | 0.1850 |
| 4-WZR-M-OD V1 | 0.39 | 0.5874 | | |
| 4-WZR-M-OD V2 | 0.31 | 1.1562 | | |
| 9-MZX-F-OS V2 | 0.03 | 0.1816 | 0.4105 | −1.7418 |
| 10-LWQ-F-OD V2 | −0.01 | 0.3343 | 0.5006 | −2.2028 |
| 10-LWQ-F-OS V2 | 0.08 | 0.2861 | 0.1075 | −0.5130 |

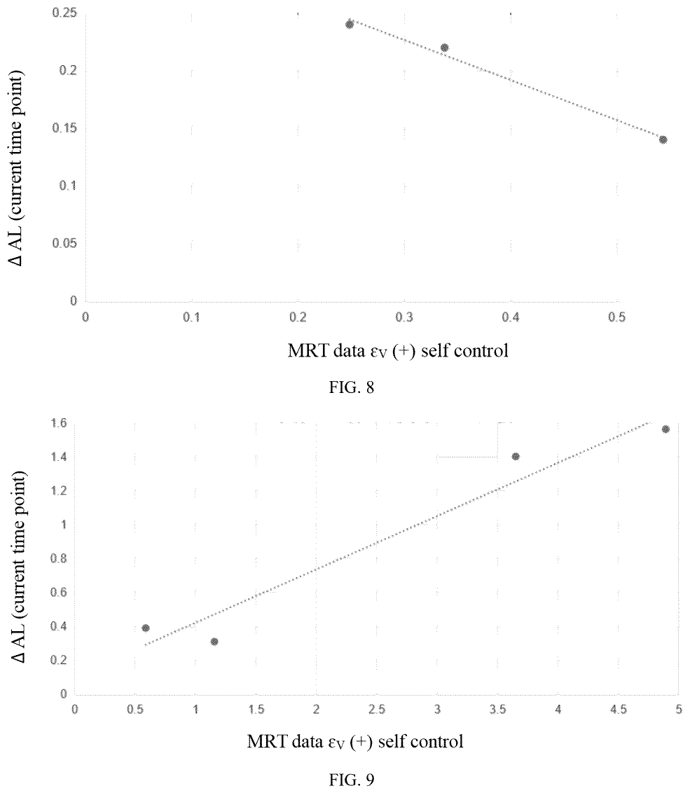

FIG. 8 shows a linear relation between volumetric strain of a retinal photofocal plane (self control) and eye axial variation at the current time point of a nearsighted person. FIG. 9 shows a linear relation between volumetric strain of a retinal photofocal plane (self control) and eye axial variation at the current time point of a farsighted person. FIG. 11 shows a linear relation between volumetric strain variation of a retinal wavefront and eye axial variation at the current time point of a nearsighted person. FIG. 13 shows a linear relation between volumetric strain variation of a retinal wavefront and eye axial variation at the current time point of a farsighted person.

Referring to FIG. 5, it shows a schematic structural diagram of a monitoring system for an eyeball emmetropization process provided by the present application. The monitoring system for an eyeball emmetropization process includes: an acquisition module, configured to acquire eyeball data information about an eyeball to be monitored; a calculation module, configured to calculate retinal optical characteristic data information about the eyeball to be monitored on the basis of the eyeball data information; an analysis module, configured to analyze and process the retinal optical characteristic data information to obtain an equation representing individual emmetropization characteristics as well as related parameters; and an output module, configured to output the results of monitoring and predicting the refractive development of the eyeball to be monitored.

In an embodiment of the present application, the acquisition module acquires corresponding eyeball data information after measuring the eyeball to be monitored by a medical measuring instrument, and the calculation module calculates corresponding retinal optical characteristic data information by a preset mathematical model according to the acquired eyeball data information. Specifically, the volumetric strain of the retinal photofocal plane or retinal wavefront of the eyeball is calculated to reflect the variation in the retinal optical characteristics in the refractive status-guided emmetropization process of the eyeball. The mathematical model has the following formula:

$${ε}_{w} ( φ , θ ; T ) = \frac{1}{3} \{ 3 {\overline{H}}_{1} ( φ ; 0 ) \cdot [ h ( φ , θ ; T ) ] + {\overline{H}}_{2} ( φ ; 0 ) \cdot {[ h ( φ , θ ; T ) ]}^{2} \}$$

where ϵ_{w} represents volumetric strain of optical imaging of an eyeball, H₁ represents an average curvature of optical imaging of an eyeball, H₂ represents a Gaussian curvature of optical imaging of an eyeball, h represents an elevation difference of optical imaging of an eyeball, φ and θ represent a latitude and a longitude in the retinal photofocal plane or the retinal wavefront, respectively, and T represents a time point.

An embodiment of the present application further provides a computer-readable storage medium having at least one computer program stored thereon. The computer program is employed to implement the monitoring method for an eyeball emmetropization process as described above.

An embodiment of the present application further provides a computer program product, which, when runs on a computer, causes the computer to perform the monitoring method for an eyeball emmetropization process as described above.

An embodiment of the present application further provides a processing terminal including a memory and a processor. The memory stores with a processing program, which, when executed by the processor, implements the steps of the monitoring method for an eyeball emmetropization process as described above.

The above are only the specific implementations of the present application. The above scenarios are only as examples, and do not constitute a limitation on the application scenarios of the technical solutions provided by the embodiments of the present application, and the technical solutions of the present application can be applied to other scenarios. Any variations or replacements that can easily be thought of by any skilled familiar with the technical field within the technical scope disclosed by the present application shall be covered by the protection scope of the present application. Therefore, the technical solutions provided by the embodiments of the present application are applicable to similar technical problems.

In the present application, the same or similar term concepts, technical solutions and/or application scenario are generally described in detail only at the first occurrence, and are not repeated for the sake of simplicity when they are repeated later, and in understanding the technical solutions and other contents of the present application, the same or similar term concepts, technical solutions and/or application scenario, etc., which are not described in detail later, can be referred to the previous detailed descriptions.

1. A monitoring method for an eyeball emmetropization process, comprising:
acquiring eyeball data information about an eyeball to be monitored;
outputting retinal optical characteristic data information about the eyeball to be monitored on the basis of the eyeball data information about the eyeball to be monitored; and
analyzing and processing the retinal optical characteristic data information about the eyeball to be monitored, and outputting, for the eyeball to be monitored, an equation representing individual emmetropization characteristics as well as related parameters.

2. The monitoring method according to claim 1, wherein the acquiring eyeball data information about an eyeball to be monitored comprises the steps of:
measuring the eyeball to be monitored by means of a medical measuring instrument;
storing the measured eyeball data information on the basis of user information about the eyeball to be monitored; and
acquiring, from the stored eyeball data information, data information needs to be monitored currently about the eyeball to be monitored.

3. The monitoring method according to claim 1, wherein the outputting retinal optical characteristic data information about the eyeball to be monitored on the basis of the eyeball data information about the eyeball to be monitored comprises the steps of:
inputting the data information about the eyeball to be monitored into a preset mathematical model; and
generating and outputting, after computational processing by the preset mathematical model, volumetric strain data and related values for a retinal photofocal plane or a retinal wavefront of the eyeball to be monitored.

4. The monitoring method according to claim 3, wherein the analyzing and processing the retinal optical characteristic data information about the eyeball to be monitored, and outputting, for the eyeball to be monitored, an equation representing individual emmetropization characteristics as well as related parameters comprises the steps of:
mathematically analyzing the volumetric strain data of the retinal photofocal plane or the retinal wavefront of the eyeball to be monitored and eye axial variation data, and obtaining the equation representing individual emmetropization characteristics as well as the related parameters; and
monitoring and predicting the refractive development of the eyeball to be monitored on the basis of the equation representing individual emmetropization characteristics as well as the related parameters.

5. A monitoring method for an eyeball emmetropization process, comprising:
acquiring measured data information about an eyeball to be monitored;
processing the measured data information about the eyeball to be monitored, and outputting retinal optical characteristic data information about the eyeball to be monitored; and
analyzing and processing the retinal optical characteristic data information and eye axial data information about the eyeball to be monitored, and outputting, for the eyeball to be monitored, an equation representing individual emmetropization characteristics as well as related parameters.

6. The monitoring method according to claim 5, wherein the acquiring measured data information about an eyeball to be monitored comprises the steps of:
measuring the eyeball to be monitored by means of a medical measuring instrument;
storing the measured data information on the basis of user information about the eyeball to be monitored; and
acquiring, from the stored eyeball data information, data information needs to be monitored currently about the eyeball to be monitored.

7. The monitoring method according to claim 6, wherein the outputting retinal optical characteristic data information about the eyeball to be monitored on the basis of the eyeball data information about the eyeball to be monitored comprises the steps of:
inputting the data information into a preset mathematical model; and
generating and outputting, after computational processing by the preset mathematical model, volumetric strain and related values for a retinal photofocal plane or a retinal wavefront of the eyeball to be monitored.

8. A monitoring system for an eyeball emmetropization process, comprising:
an acquisition module, configured to acquire eyeball data information about an eyeball to be monitored;
a calculation module, configured to calculate retinal optical characteristic data information about the eyeball to be monitored on the basis of the eyeball data information;
an analysis module, configured to analyze and process the retinal optical characteristic data information to obtain an equation representing individual emmetropization characteristics as well as related parameters, and to monitor and predict the refractive development of the eyeball to be monitored on the basis of the equation representing individual emmetropization characteristics as well as the related parameters; and
an output module, configured to output the results of monitoring and predicting the refractive development of the eyeball to be monitored.

9. A computer-readable storage medium, having at least one computer program stored thereon, wherein the computer program is employed to implement a monitoring method for an eyeball emmetropization process according to any one of claims 1-4 and/or claims 5-7.

10. A computer program product, which, when runs on a computer, causes the computer to implement a monitoring method for an eyeball emmetropization process according to any one of claims 1-4 and/or claims 5-7.
